(19) United States
(12) Patent Application Publication
(10) Pub. No.: US 2014/0154334 A1
(43) Pub. Date: Jun. 5, 2014

(54) TOXIN DECONTAMINANT FOOD PRODUCT AND METHOD OF TREATING DISORDERS OF THE GASTROINTESTINAL TRACT

(71) Applicant: DENOVO INC., Baltimore, MD (US)

(72) Inventor: Michael A. STANG, Baltimore, MD (US)

(73) Assignee: DENOVO INC., Baltimore, MD (US)

(21) Appl. No.: 14/170,921

(22) Filed: Feb. 3, 2014

Related U.S. Application Data
(63) Continuation of application No. 13/671,851, filed on Nov. 8, 2012, now abandoned.
(60) Provisional application No. 61/557,510, filed on Nov. 9, 2011.

(51) Int. Cl. A23L 1/304; A61K 33/44

(52) U.S. Cl. CPC A23L 1/304; A61K 33/44
USPC 424/600

(57) ABSTRACT

The invention encompasses antidotal food products containing activated charcoal for ingestion into the gastrointestinal tract of a user for treating various gastrointestinal disorders. The antidotal food product is orally administered having the visual appearance and physical properties which enables the desire on the part of young children to chew and ingest the food product.

The present application is a continuation application of U.S. application Ser. No. 13/671,851, filed Nov. 8, 2012, which claims benefit of U.S. Provisional Patent Application No. 61/557,510, filed Nov. 9, 2011, the disclosures of each of which are incorporated by reference herein in their entireties.

### FIELD OF THE INVENTION

The invention encompasses antidotal food products containing activated charcoal for ingestion into the gastrointestinal tract of a user for treating various gastrointestinal disorders. The antidotal food product is orally administered having the visual appearance and physical properties which enables the desire on the part of young children to chew and ingest the food product.

### BACKGROUND OF THE INVENTION

Poisonings by the ingestion of toxic substances have historically been and continue to be a significant problem today. With the ongoing proliferation of an expansive variety of commercially-available household products, access to a plethora of toxic substances just in the average home is currently at or near an all-time high. While efforts in recent years, such as conspicuous labeling, tamper-proof sealing campaigns, and limiting the number of tablets in bottles of children's medicines as well as a concerted informational program appear to have been successful in preventing dramatic growths in the number of poisoning incidents that occur annually, significant numbers of poisoning incidents continue to occur, an overwhelming percentage of those incidents occurring at a residence, and the majority of the victims being young children. In 1993 alone, for instance, there were 1,751,476 human poisoning cases reported to recognized poison centers, over 90% of which occurred at a residence, according to the 1993 Annual Report of the American Association of Poison Control Centers Toxic Exposure Surveillance System. Children under the age of six made up approximately 56% of the victims in these cases. Considering that only about 70% of the American population actually had access at the time to a poison center recognized in the study, the number of incidents that actually occurred in 1993 is likely to have been significantly higher than these numbers would indicate, with the total number of cases more realistically having been on the order of 2.5 million.

In the emergency treatment of poisoning victims, the effort centers around two main objectives: general support and stabilization of the victim, and decontamination of the victim. As decontamination and treatment must begin immediately in such toxicologic emergencies, often without the benefit of full and thorough clinical information on the patient, it is particularly important that any drug or therapeutic substance administered to the patient be substantially free of unwanted side effects that may cause unforeseen complications, or even death. For this reason, sorbents which, when introduced into the patient's gastro-intestinal tract, resist decomposition and adsorb the ingested toxins until eventual excretion by the patient have been employed for decontamination. Of those sorbents, activated charcoal has emerged in the field of emergency toxicological treatment as the decontaminant of choice, its routine use in the treatment of poison victims did not become widespread until the 1980s; however, its administration to poisoning victims has now surpassed the administration of syrup of ipecac as the single most important toxicological treatment measure.

Activated charcoal is a fine, black, powdery substance which is tasteless, odorless, and non-toxic. Activated charcoal is generally formed by oxidation (activation) of combustion residue derived from a controlled combustion process performed on wood, peat, or another organic material. The oxidation and controlled combustion steps combine to yield a substance composed of extremely porous particles which give it extraordinarily high internal surface area, typically ranging between 900 and 2000 m.sup.2/g. Due to its extraordinary surface area, activated charcoal exhibits great adsorptivity and, thus, has proven to be quite effective as a decontaminant when introduced in sufficient quantities into the gastro-intestinal tract of a poisoning victim. The highly adsorptive activated carbon particles within the gastro-intestinal tract are capable of adsorbing toxin, not only from the contents of the gastro-intestinal tract but even from the blood stream (by “intestinal dialysis”) through the blood vessels which supply the gastro-intestinal tract. These toxins, now bound to the activated charcoal, are excreted in the stool.

Activated charcoal is currently available in several forms to be orally administered to poisoning victims. In the most widely used form, activated charcoal is contained in a liquid suspension such as the commercially-available Actidose Aqua and Charcoaid 2000 suspensions. Activated charcoal is also available extensively in Europe, and to a more limited extent in the United States, simply in its powdered or granulated form for mixture within a drinkable liquid prior to ingestion. In yet another form, activated charcoal is contained in over-the-counter tablets or capsules widely available in Europe for the treatment of gas and upset stomach. Use of these tablets or capsules for decontamination in toxicological treatment, however, is not readily feasible. Even if all active ingredients other than activated charcoal were removed therefrom, the relatively high dosages required in most poisoning incidents would necessitate the ingestion of many such tablets or capsules, a daunting task even for the average adult, let alone for the average young child.

In whatever form activated charcoal is delivered to the gastro-intestinal tract, suspended in a liquid, compressed within a tablet or capsule, or simply in its raw powdery state, the activated charcoal is likely to have significantly beneficial, if not life-saving, effects on the poisoning victim—that is, if it can be properly delivered in the necessary quantities to the gastro-intestinal tract of that victim. Therein lies the single greatest obstacle to optimal utilization of activated charcoal as a decontaminant in toxological treatment. Each of the currently available forms in which activated charcoal is available for oral ingestion utterly fails to adequately induce or at least encourage proper ingestion of a sufficient dose of the activated charcoal by the victim. Essentially, except in the tablet or capsule form (which presents its own obstacles to ingestion), the antidotal substances are extremely unpalatable and, in fact, quite noxious. Liquid antidotal suspensions containing activated charcoal, for instance, form a black, gritty liquid bearing a striking resemblance to expended engine crankcase oil and lacks the pleasant taste which, theoretically, might cause the ingesting individual to even momentarily forget the unpleasant appearance, texture, and consistency of that which he or she is ingesting.

### SUMMARY OF THE INVENTION

The invention encompasses antidotal food products containing activated charcoal for use as an antidotal product, which includes the necessary palatability not heretofore seen in prior antidotes. In one embodiment, the subject antidotal food product is in the form of a readily recognizable cookie sandwich having a pair of black wafers sandwiching a creamy filling. The subject antidotal food product in this form has the appearance, weight, and feel of very common and popular cookie treats. To the average young child, the subject antidotal food product is, thus, indistinguishable from the delectable cookie treats he or she is accustomed to eating.

The subject antidotal food product also incorporates a number of component compositions, which give it a pleasant flavor and a texture not unlike that of the commonly-available cookie treats. Thus, a child would not only be enticed to place the subject antidotal food product into his or her mouth, he or she would actually be encouraged thereafter to chew and swallow the product. The pleasant taste would encourage the child to so ingest additional portions of the product if necessitated by the required activated charcoal dosage.

Given its inherent palatability, the subject antidotal food product could easily be administered in the home or any other setting outside a medical institution. Hence, the subject antidotal food product would not only expand the usage of activated charcoal as a decontaminant, it would actually enhance in a significant manner the effectiveness of that usage.

While the subject antidotal food product successfully incorporates with activated charcoal other component compositions, which combine therewith to yield heretofore unsurpassed palatability without diminishing the decontaminating function of the activated charcoal; it must be recognized that due to the properties and characteristics of activated charcoal, developing the proper combinations was hardly a trivial task. One cannot simply blend a pleasant tasting composition with activated charcoal to form a useful antidotal product. Many substances will tend in varying degrees to bind with the activated charcoal particles and significantly diminish, even completely nullify its adsorptivity. An end product would then result which is either devoid of a decontaminating capability, or which requires the ingestion of much too great a quantity to be of any practical use.

Realization of the unique combination of component compositions that essentially form the subject antidotal food product was the result of extensive testing and research. That testing and research is described in great detail in following paragraphs.

The invention further encompasses methods of treating a subject suffering from a gastrointestinal disorder comprising administering the antidotal food products containing activated charcoal to a subject in need thereof. Gastrointestinal disorders encompassed by the methods of the invention include diarrhea, intestinal gas, abdominal/enteric sepsis, peptic ulcer disease, gastritis, reflux esophagitis, food poisoning, bad breath, gingivitis, and ostomy odor.

In another embodiment, the invention encompasses antidotal food products containing activated charcoal for use binding dietary fat.

The invention encompasses an edible, toxin-decontaminant product that comprises a therapeutically or prophylactically effective amount of activated charcoal, which is useful in mitigating, substantially reducing, or causing the cessation of at least one adverse effect in a subject that ingested a toxic or poisonous substance or ingested a substance causing illness.

In one embodiment, the invention encompasses an edible, toxin-decontaminant product comprising a plurality of ingredients, which comprises activated charcoal.

In another embodiment, the invention encompasses an edible, toxin-decontaminant product comprising activated charcoal and comprising one or more flavoring agents.

In another embodiment, the invention encompasses an edible, toxin-decontaminant product comprising activated charcoal and comprising one or more complexing or thickening agents.

In another embodiment, the invention encompasses an edible, toxin-decontaminant product comprising activated charcoal and comprising one or more emulsifying agents.

In another embodiment, the invention encompasses an edible, toxin-decontaminant product comprising activated charcoal and comprising one or more agents to improve porosity and texture.

In another embodiment, the invention encompasses an edible, toxin decontaminant product comprising activated charcoal and comprising water,

In another embodiment, the invention encompasses an edible, toxin-decontaminant product comprising activated charcoal wherein the ingredients are processed to substantially remove water, for example, by heat to produce the product.

In another embodiment, the invention encompasses an edible, toxin-decontaminant product comprising activated charcoal and a plurality of ingredients optionally comprising one or more flavoring agents; optionally comprising one or more complexing or thickening agents; optionally comprising one or more emulsifying agents; water; optionally comprising ammonia, and optionally comprising an agent to improve porosity and texture, wherein the ingredients are processed to substantially remove water, for example, by heat to produce the product.

In another embodiment, the invention encompasses an edible, toxin decontaminant product comprising activated charcoal and a plurality of ingredients comprising one or more flavoring agents; one or more complexing or thickening agents; one or more emulsifying agents; water; optionally ammonia, and an agent to improve porosity and texture, wherein the ingredients are processed to substantially remove water, for example, by heat to produce the product.

In another embodiment, the invention encompasses a method of producing an edible, toxin-decontaminant product, which comprises activated charcoal and a plurality of ingredients comprising one or more flavoring agents; one or more complexing or thickening agents; and one or more emulsifying agents; optionally ammonia, adding water to produce a first mixture, for instance, a dough; adding an agent to improve porosity and texture, blending to produce a second mixture; and baking the second mixture to produce the product.

In another embodiment, the invention encompasses a gastrointestinal decontaminant produced by the steps of combining activated, charcoal and a plurality of ingredients comprising one or more flavoring agents; one or more complexing or thickening agents; and one or more emulsifying agents; adding water to produce a first mixture, for instance, a dough; adding a porosity or texturing agent, and processing the ingredients to substantially remove water to produce a first product; blending together one or more flavoring agents, lecithin, salt, sugar, and shortening to produce a filling mixture composition; and sandwiching said filling mixture composition between two of said first product.

In yet another embodiment, the invention encompasses an edible, toxin-decontaminant product that contains a therapeutically or prophylactically effective amount of activated charcoal and one or more second active agents, which is useful in mitigating, substantially reducing, or causing the cessation of at least one adverse effect in a subject associated with the ingestion of a toxic or poisonous substance or the ingestion of a substance causing illness.

### DETAILED DESCRIPTION OF THE INVENTION

As used herein and unless otherwise indicated, the term “baked” or “baking” refer to placing the combined ingredients that compose the edible product in an oven or other chamber at a defined temperature and allowing the ingredients to produce the product, for instance a food product, for a defined amount of time. The temperature at which the ingredients are “baked” or the “baking” refers to the oven temperature and does not mean heating the actual ingredients to this temperature.

As used herein and unless otherwise indicated, the term “causing the cessation of adverse effects” advantageous partial or complete conclusion or termination of at least one adverse effect associated with the ingestion of a poison or toxin. In an illustrative embodiment, the term “causing the cessation of adverse effects” refers to the partial or complete conclusion or termination of more than one adverse effect associated with the ingestion of a poison or toxin. In a particular illustrative embodiment, the term “causing the cessation of adverse effects” refers to the partial or complete conclusion or termination of all adverse effects associated with the ingestion of a poison or toxin.

As used herein and unless otherwise indicated, the term “coloring agents” are agents that give the edible product a more pleasing appearance, and in addition help the manufacturer to control the product during its preparation and help the user to identify the product. Any of the approved certified water-soluble FD&C dyes, mixtures thereof, or their corresponding lakes may be used. A color lake is the combination by adsorption of a water-soluble dye to a hydrous oxide of a heavy metal, resulting in an insoluble form of the dye. In other embodiments, coloring agents used outside the food industry, for example, pharmaceutical coloring agents, may be used.

As used herein and unless otherwise indicated, the term “complexing agent” and “thickening agent” are used interchangeably and refer to any substance that increases the viscosity of the product. As used herein, these terms included binding agents or binders. Examples of “complexing agents” and “thickening agents” include, but are not limited to, sodium stearoyl lactylate, modified food starch, Inscosity, and high fructose corn syrup. In other embodiments, agents used outside of the food industry for instance pharmaceutical additives, such as PVP, may be used.

As used herein and unless otherwise indicated, the term “dough” refers to a mixture, preferably a thick, soft mixture of one or more materials, preferably dry materials, which may include flour and one or more liquids.

As used herein and unless otherwise indicated, the term “edible product” refers to an edible composition of the invention containing a therapeutically or prophylactically effective amount of activated charcoal. Examples of illustrative embodiments of the edible product include, but are not limited to, breads, cakes, muffins, pastries, or cookies containing a therapeutically or prophylactically effective amount of activated charcoal.

As used herein and unless otherwise indicated, the term “effective amount” means an amount of activated charcoal that is sufficient to provide the desired local or systemic effect and performance at a reasonable benefit/risk ratio attending any medical treatment

As used herein and unless otherwise indicated, the term “emulsifying agent” refers to a substance having both hydrophilic and hydrophobic character that acts to stabilize an emulsion by coating particles of a dispersed phase and preventing coagulation of colloidal particles. In additional illustrative embodiments, agents used outside of the food industry, for instance, pharmaceutical additives may be used. Examples of “emulsifying agents” include, but are not limited to, monoglycerides and diglycerides other embodiments, agents used outside of the food industry for instance pharmaceutical additives may be used.

As used herein and unless otherwise indicated, the term “fat replacer” refers to a substance that attempts to recreate the attributes of fat, while also significantly reducing fat and calorie content. In other embodiments, agents used outside of the food industry, for instance, pharmaceutical additives may be used.

As used herein and unless otherwise indicated, the terms “gastrointestinal decontaminant,” and “toxin decontaminant” are used interchangeably and refer to an edible product of the invention containing activated charcoal.

As used herein and unless otherwise indicated, the term “ingestion” refers to oral consumption.

As used herein and unless otherwise indicated, the term “mitigate” or “mitigate adverse effects” are used interchangeably and refer to making less severe, intense, harsh, rigorous, painful or ameliorate at least one adverse effect associated with the ingestion of a toxin, poison or substance causing illness in a subject.

As used herein and unless otherwise indicated, the term “palatable” and “edible” are used interchangeably and refer to the ability of a subject to orally ingest an edible product of the invention.

As used herein and unless otherwise indicated, the term “pharmaceutically acceptable” refers to materials and compositions that are physiologically tolerable and do not typically produce a severe allergic or similar untoward reaction, such as gastric upset, dizziness and the like, when administered to a human. Typically, as used herein, the term “pharmaceutically acceptable” means approved by a regulatory agency of the Federal or a state government or listed in the U.S. Pharmacopeia or other generally recognized pharmacopeia for use in animals, and more particularly in humans.

As used herein and unless otherwise indicated, the terms “poisonous substance,” “poison,” “toxin,” and “toxic substance” are used interchangeably and refer to a chemical that adversely affects health by causing injury, illness, or death. These terms further include, but are not limited to, any substance, which is harmful to living tissue when ingested orally. Determining factors include concentration, exposure time, particle size, the substance's affinity for tissue and sensitivity of the exposed tissue to that substance. Examples of poisonous substances, poisons, toxins or toxic substances include, but are not limited to, cleaning products (e.g., bleach, detergent, floor cleaner, furniture polish); cosmetics (e.g., nail polish, nail polish remover, make-up), perfumes, plants, pesticides (e.g., bug killers, weed killers, lawn products), prescription or non-prescription drugs.

As used herein and unless otherwise indicated, the term “processed to substantially remove water” refers to any method acceptable in the food industry to remove at least a portion of water, for example to remove from about 10% to about 90%, from about 20% to about 80%, from about 30% to about 70%, from about 40% to about 60% of water in a mixture. In some illustrative embodiments, the processed product will retain or comprise about 2% to about 35% water, about 8% to about 30% water, about 13% to about 25% water, or about 18% to about 22% water. Such methods include, but are not limited to, baking—as defined herein, frying, desiccation (i.e., drying) or any other means available.

As used herein and unless otherwise indicated, the term “prophylactically effective” refers to an amount of activated charcoal capable of mitigating or substantially reducing adverse effects associated with the ingestion of a toxin or poison. In one embodiment, the edible product of the invention is administered as a preventative measure to a subject, preferably a human, who potentially ingested a toxin or poison. Accordingly, the compositions of the invention may be used for the prevention of at least one adverse effect and concurrently treating another (e.g., prevention of adverse effects of poison ingestion while treating emesis or increased heart rate).

As used herein and unless otherwise indicated, the term “sorbent” refers to any material possessing adsorptive power useful in adsorbing a poison to any degree. Example of illustrative sorbents of the invention include, but are not limited to, activated charcoal or AST-120, which is highly adsorptive, spherical carbon microspheres or combinations thereof. In certain embodiments, the sorbent of the invention is activated charcoal.

As used herein and unless otherwise indicated, the phrase “substance causing illness” refers to any substance that caused a detrimental effect and induces substances that may act in a therapeutic or prophylactic manner in minute levels, but become toxic when the quantity is larger. Examples of substances causing illness include, but are not limited to, nutrients, therapeutic drugs, vitamins, minerals, herbs, prophylactic drugs. Exemplary not limiting examples of substance(s) causing illness include, but are not limited to, St. John's wart, saw palmetto, acetaminophen, aspirin, adriamycin, alcohol, amiodarone, chloramphenical, cisplatin, dapsone, dilantin, disulfuram, glutethimide, hydrlazine, isoniazid, vitamin A, vitamin D, vitamin B6, metronidazole, nitrofurantoin, furadantin, macrodantin, penicillin, perhexyline, taxol, vincristine, or zoloft, misomidazole, or lithium.

As used herein, the term “substantially reduces” refers to the ability of an edible product of the invention to measurably reduce the effects of at least one adverse effect associated with ingestion of a toxin or poison. In a preferred embodiment, substantially reduces refers to the ability of an edible product of the invention to reduce all measurable adverse effects associated with ingestion of a toxin or poison.

As used herein, the term “subject” can be a human, a mammal, or an animal. The subject being treated is a patient in need of treatment, preferably a human. In many instances, the subject is a child.

As used herein, the terms “surface area” and “internal surface area” may be used interchangeably.

As used herein and unless otherwise indicated, the term “therapeutically effective” refers to an amount of activated charcoal able to cause an amelioration or substantial reduction of at least one adverse effect associated with the ingestion of a toxin or poison, or at least one discernible symptom thereof. “Therapeutically effective” also refers to an amount of activated charcoal to result in an amelioration of at least one measurable physical parameter, not necessarily discernible by the patient. In yet another embodiment, the term “therapeutically effective” refers to an amount of an activated charcoal to inhibit the progression of at least one adverse effect, either physically (e.g., stabilization of a discernible symptom), physiologically (e.g., stabilization of a physical parameter), or both. In yet another embodiment, the term “therapeutically effective” refers to an amount of activated charcoal resulting in a delayed onset of a disease or disorder. The amount of activated charcoal, which constitutes a “therapeutically effective amount” will vary depending on the toxin or poison ingested, the severity of the condition, and the age and body weight of the subject to be treated, but can be determined routinely by one of ordinary skill in the art having regard to his/her own knowledge and to this disclosure.

The invention encompasses an edible product, such as a food product, containing a therapeutically or prophylactically effective amount of activated charcoal. In an illustrative embodiment, the edible product is in the form of a snack (e.g., a cookie sandwich or pastry product), which is perceived as palatable, for example, by children. The edible product of the invention generally exhibits the appearance, the texture, the friability, and the sweet flavor, which typically characterize snack products. In a particular embodiment, the edible product is in the form of two wafers sandwiching a cream. However, one of ordinary skill in the art will readily recognize that the edible product of the invention can resemble any dessert product including, but not limited to, a candy product, candy bar, cupcake, cookie, a wafer, a pie, a pastry, a health food bar or a donut. Optionally, the product may have a coating of material including, but not limited to, chocolate or sugar-based glaze. In addition, the product can optionally contain flavor bits or inclusions such as, but not limited to, jimmies, flavor nuggets, cookie pieces, and chocolate chips to enhance flavor, texture, and appearance.

In one embodiment, the invention encompasses an edible, toxin-decontaminant product comprising a plurality of ingredients, which comprises activated charcoal, wherein the ingredients are processed by heat or drying to produce the product.

in another embodiment, the invention encompasses an edible, toxin-decontaminant edible product comprising a plurality of ingredients including one or more of the following: (i) one or more flavoring agents; (ii) one or more complexing or thickening agents; (iii) one or more sorbents, for example, activated charcoal or AST-120; (iv) one or more emulsifying agents; (v) water; and (vi) soy protein crisps; wherein the ingredients are processed to substantially remove water, for example, by baking or frying to produce the product.

In another embodiment, the invention encompasses an edible, toxin-decontaminant edible product comprising a plurality of ingredients including one or more of the following: (i) one or more flavoring agents; (ii) one or more complexing or thickening agents; (iii) activated charcoal; (iv) one or more emulsifying agents; (v) water; and (vi) soy protein crisps; wherein the ingredients are processed to substantially remove water, for example, by baking or frying to produce the product.

In an illustrative embodiment, the activated charcoal is characterized by an internal surface area of from about 800 m²/g to about 3,000 m²/g; in another illustrative embodiment, the activated charcoal is characterized by an internal surface area of from about 1500 m²/g to about 2,500 m²/g; in yet another illustrative embodiment, the activated charcoal is characterized by an internal surface area of about 2,000 m²/g. However, any ingestible activated charcoal may be used for example the activated charcoal in the product may be in one or more forms such as powder, granules, or brittle chips and can vary in source material, pore size distribution, and adsorptivity. In addition, the product can contain more than one type of activated charcoal.

In another illustrative embodiment, the product includes activated charcoal in an approximate weight range of from about 20% to about 80% thereof. In another illustrative embodiment, the product includes activated charcoal in an approximate weight range of from about 30% to about 70% thereof. In another illustrative embodiment, the product includes activated charcoal in an approximate weight range of from about 45% to about 65% thereof. In another illustrative embodiment, the product includes activated charcoal in an approximate weight of about 60% thereof.

In another embodiment, the flavoring agent is vanilla flavor, chocolate flavor, cocoa, salt, sugar, or a sweetener or combinations thereof; although, any flavoring agent can be used. In an illustrative embodiment, the flavoring agent is in the range of from about 0.001% to about 15%. In a particular illustrative embodiment, the product includes vanilla flavor in the approximating weight range of about 0.01% to about 1% thereof. In yet another illustrative embodiment, the product includes the vanilla flavor in the approximating weight of about 0.05%. In another particular illustrative embodiment, the product includes chocolate flavor in an approximate weight range of about 0.5% to about 3% thereof. In yet another illustrative embodiment, the product includes chocolate flavor in the approximate weight of about 2% thereof. In another particular illustrative embodiment, the product includes salt in an approximate weight range of about 0.1% to about 1% thereof. In an illustrative embodiment, the product includes the salt in the approximating weight of about 0.4% thereof. In another particular illustrative embodiment, the flavoring agent is sweetener in the approximate weight range of about 0.001% to about 10% thereof. In another particular illustrative embodiment, the product includes sweetener in an approximate weight range of about 0.001% to about 5% thereof. In an illustrative embodiment, the product includes a sweetener in the approximate weight of about 2% thereof. In another particular illustrative embodiment, the sweetener present in the product is neotame in the approximate weight range of about 0.001% to about 1.5% thereof. In another particular illustrative embodiment, the product includes sugar in the approximating weight range of about 2% to about 6% thereof. In yet another illustrative embodiment, the product includes the sugar in the approximating weight of about 4.5% thereof. One of ordinary skill in the art will readily understand that the amount of sweetener used is dependent on the specific sweetener. Illustrative examples of sweeteners that can be used in an embodiment of the invention include, but are not limited to, aspartame, sucralose or neotame or mixtures thereof.

In another embodiment, the complexing or thickening agent is sodium stearoyl lactylate or modified food starch or combinations thereof although any complexing or thickening agent may be used. In an illustrative embodiment, the product includes the complexing or thickening agent in an approximate weight range of about 0.5% to about 4% thereof. In a particular illustrative embodiment, the product includes sodium stearoyl lactylate in an approximate weight range of about 0.1% to about 2% thereof. In another particular illustrative embodiment, the product includes sodium stearoyl lactylate in the approximate weight of about 0.9% thereof. In another particular illustrative embodiment, the product includes modified food starch in the approximate weight range of about 0.1% to about 2% thereof. In another particular illustrative embodiment, the product includes the modified food starch in the approximate weight of about 0.9% thereof.

In another embodiment, the emulsifying agent is a mono or diglyceride; although or combinations thereof, any emulsifying agent may be used. In an illustrative embodiment, the product includes the emulsifying agent in an approximate weight range of about 0.5% to about 4% thereof. In a particular illustrative embodiment, the product includes an emulsifying agent in the approximate weight range of about 1% to about 3% thereof. In yet another illustrative embodiment, the product includes the emulsifying agent in the approximate weight of about 2% thereof.

In another illustrative embodiment, the product includes water in the approximating weight range of about 15% to about 60% thereof. In a particular illustrative embodiment, the product includes the water in the approximate weight of about 25% thereof.

In another embodiment, the agent to improve porosity and texture is soy protein crisp or another protein product such as a rice protein crisp or is glycerine or combinations thereof. In an illustrative embodiment, the product includes the agent to improve porosity and texture in an approximate weight range of about 5% to about 15% thereof. In a particular illustrative embodiment, the product includes soy protein crisp rice in the approximate weight range of about 5% to about 15% thereof. In another particular illustrative embodiment, the product includes the soy protein crisp rice in the approximate weight of about 11% thereof. In another particular embodiment, the product includes glycerine in the approximate weight range of about 6% to about 8% thereof.

In one embodiment, the baking is done at an oven temperature of from about 250 .degree. F. to about 450 .degree. F., wherein the combined ingredients are placed in the oven for a time of from about 5 to about 35 minutes. In another embodiment, the combined ingredients are placed in the oven at an oven temperature of about 350 .degree. F. for a time of about 5 to about 15 minutes. In yet another embodiment, the baking is done at an oven temperature of about 350 .degree. F. for a time of about 10 minutes.

In another embodiment, the invention encompasses a method of producing an edible, toxin-decontaminant product comprising:

(a) combining a plurality of ingredients including:
i. optionally one or more flavoring agents;
ii. optionally one or more complexing or thickening agents;
iii. activated charcoal; and
iv. optionally one or more emulsifying agents;

(b) blending the components to produce a first mixture;

(c) optionally adding water to produce a mixture, for instance, a dough;

(d) optionally adding soy protein crisp rice or rice crisp and blending to produce a second mixture; and

(e) baking the second mixture to produce the product.

In many embodiments, the instances as described herein may produce a dough.

In an illustrative embodiment, the activated charcoal is characterized by an internal surface area of from about 800 m²/g to about 3,000 m²/g; in another illustrative embodiment, the activated charcoal is characterized by an internal surface area of from about 1500 m²/g to about 2,500 m²/g; in yet another illustrative embodiment, the activated charcoal is characterized by an internal surface area of about 2,000 m²/g.

In another illustrative embodiment, the method includes activated charcoal in an approximate weight range of from about 20% to about 80% thereof. In another illustrative embodiment, the method includes activated charcoal in an approximate weight range of from about 25% to about 75% thereof. In another illustrative embodiment, the method includes activated charcoal in an approximate weight range of from about 30% to about 70% thereof.

In another embodiment, the flavoring agent is vanilla flavor, chocolate flavor, salt, sugar, or a sweetener or combinations thereof, although any flavoring agent can be used. In an illustrative embodiment, the method includes vanilla flavor in the approximate weight range of about 0.002% to about 15% thereof. In another illustrative embodiment, the method includes the vanilla flavor in the approximate weight of about 0.01% to about 1%. In yet another illustrative embodiment, the method includes the vanilla flavor in the approximate weight of about 0.05%. In another illustrative embodiment, the method includes chocolate flavor in an approximate weight range of about 0.5% to about 3% thereof. In yet another illustrative embodiment, the method includes chocolate flavor in the approximate weight of about 2% thereof. In an illustrative embodiment, the method includes salt in an approximate weight range of about 0.1% to about 1% thereof. In an illustrative embodiment, the method includes the salt in the approximating weight of about 0.4% thereof. In an illustrative embodiment, the method includes sugar in the approximate weight range of about 2% to about 6% thereof. In yet another illustrative embodiment, the method includes the sugar in the approximate weight of about 4.5%. In an illustrative embodiment, the method includes sweetener in an approximate weight range of about 0.001% to about 10% thereof. In an illustrative embodiment, the method includes a sweetener in the approximate weight of about 2% thereof. One of ordinary skill in the art will readily understand that the amount of sweetener used is dependent on the specific sweetener. Illustrative examples of sweeteners that can be used in an embodiment of the invention include, but are not limited to, aspartame, sucralose or neotame or mixtures thereof.

In another embodiment, the complexing or thickening agent is sodium stearoyl lactylate or modified food starch or combinations thereof; although, any complexing or thickening agent may be used. In an illustrative embodiment, the method includes the complexing or thickening agent in an amount of about 0.5% to about 4%. In an illustrative embodiment, the method includes sodium stearoyl lactylate in an approximate weight range of about 0.1% to about 2% thereof. In yet another illustrative embodiment, the method includes sodium stearoyl lactylate in the approximate weight of about 0.9% thereof. In another illustrative embodiment, the method includes modified food starch in the approximate weight range of about 0.1% to about 2% thereof. In another illustrative embodiment, the method includes the modified food starch in the approximate weight of about 0.9% thereof.

In another embodiment, the emulsifying agent is a mono or diglyceride or combinations thereof; although any emulsifying agent can be used. In an illustrative embodiment, the method includes an emulsifying agent in the approximate weight range of about 0.5% to about 4% thereof. In a particular illustrative embodiment, the method includes an emulsifying agent in the approximate weight range of about 1% to about 3% thereof. In yet another illustrative embodiment, the method includes the emulsifying agent in the approximate weight of about 2% thereof.

In another illustrative embodiment, the method includes water in the approximate weight range of about 15% to about 60% thereof. In another illustrative embodiment, the method includes the water in the approximate weight of about 25% thereof.

In another embodiment, the agent to improve porosity and texture is soy protein crisp or another protein product such as rice crisp or is glycerine or combinations thereof. In an illustrative embodiment, the method includes the agent to improve porosity and texture in an approximate weight range of about 5% to about 15% thereof. In a particular illustrative embodiment, the method includes soy protein crisp rice in the approximate weight range of about 5% to about 15% thereof. In another particular illustrative embodiment, the method includes the soy protein crisp rice in the approximate weight of about 11% thereof. In another particular embodiment, the method includes glycerine in the approximate weight range of about 6% to about 8% thereof.

In one embodiment, the baking is done at an oven temperature of from about 250 .degree. F. to about 450 .degree. F., wherein the combined ingredients are placed in the oven for a time of from about 5 to about 15 minutes. In another embodiment, the baking is done at an oven temperature of about 350 .degree. F. to for a time of about 10 minutes.

In another embodiment, the invention encompasses a gastrointestinal decontaminant produced by the steps of:

(a) combining a plurality of ingredients including; (i) one or more flavoring agents; (iii) one or more complexing or thickening agents; (iii) one or more sorbents, for example, activated charcoal; and (iv) optionally one or more emulsifying agents;

(b) blending the components to produce a mixture;

(c) optionally adding water to produce a mixture, for instance, a dough;

(d) optionally adding an agent to increase texture or porosity to the mixture, and blending;

(e) baking the ingredients to produce a first product;

(f) optionally blending together one or more flavoring agents, lecithin, salt, sugar, and shortening to produce a filling mixture composition; and

(g) sandwiching said tilling mixture composition between two of said first product.

In an illustrative embodiment, the activated charcoal is characterized by an internal surface area of from about 800 m²/g to about 3,000 m²/g; in another illustrative embodiment, the activated charcoal is characterized by an internal surface area of from about 1500 m²/g to about 2,500 m²/g; in yet another illustrative embodiment, the activated charcoal is characterized by an internal surface area of about 2,000 m²/g.

In another illustrative embodiment, the product includes activated charcoal in an approximate weight range of from about 10% to about 80% thereof. In another illustrative embodiment, the product includes activated charcoal in an approximate weight range of from about 20% to about 60% thereof. In another illustrative embodiment, the product includes activated charcoal in an approximate weight range of from about 25% to about 45% thereof. In a particular illustrative embodiment, the product includes activated charcoal in an approximate weight of about 25% thereof.

In another embodiment, the flavoring agent is vanilla flavor, chocolate flavor, salt, sugar, or a sweetener including, but not limited to, sucrose, glucose, fructose, lactose, acesulfame-K, dextrose, sucralose, saccharin, and aspartame or neotame or combinations thereof. In an illustrative embodiment, the flavoring agent is in the range of from about 0.0001% to about 15%. In a particular illustrative embodiment, the product includes vanilla flavor in the approximate weight range of about 0.01% to about 5% thereof. In yet another illustrative embodiment, the product includes the vanilla flavor in the approximate weight of about 0.05%. In another particular illustrative embodiment, the product includes chocolate flavor in an approximate weight range of about 0.5% to about 3% thereof. In yet another illustrative embodiment, the product includes chocolate flavor in the approximate weight of about 2% thereof. In another illustrative embodiment, the product includes neotame in an amount of about 0.001% to about 1%. In another illustrative embodiment, the product includes neotame in an amount of about 0.002% to about 0.1%. In another particular illustrative embodiment, the product includes salt in an approximate weight range of about 0.1% to about 1% thereof. In an illustrative embodiment, the product includes the salt in the approximate weight of about 0.4% thereof. In another particular illustrative embodiment, the product includes sugar in the approximating weight range of about 2% to about 6% thereof. In yet another illustrative embodiment, the product includes the sugar in the approximate weight of about 4.5% thereof.

In another embodiment, the complexing or thickening agent is sodium stearoyl lactylate, modified food starch, Inscosity, or high fructose corn syrup or combinations thereof. In an illustrative embodiment, the product includes the complexing or thickening agent in an approximate weight range of about 0.5% to about 40% thereof. In a particular illustrative embodiment, the product includes sodium stearoyl lactylate in an approximate weight range of about 5% to about 30% thereof. In another particular illustrative embodiment, the product includes sodium stearoyl lactylate in the approximate weight of about 25% thereof. In another particular illustrative embodiment, the product includes modified food starch in the approximate weight range of about 0.1% to about 2% thereof. In another particular illustrative embodiment, the product includes the modified food starch in the approximate weight range of about 0.9% thereof. In another particular illustrative embodiment, the product includes high fructose corn syrup in the approximate weight range of about 5% to about 40% thereof. In another particular illustrative embodiment, the product includes the high fructose corn syrup in the approximate weight range of about 27% thereof.

In another embodiment, the emulsifying agent is a mono or diglyceride or combinations thereof. Particular examples of emulsifying agents include, but are not limited to, atmul, emplex and lecithin. In an illustrative embodiment, the product includes the emulsifying agent in an approximate weight range of about 0.5% to about 12% thereof. In a particular illustrative embodiment, the product includes an emulsifying agent in the approximate weight range of about 2% to about 9% thereof. In yet another illustrative embodiment, the product includes the emulsifying agent in the approximate weight of about 7% thereof.

In another illustrative embodiment, the product includes water in the approximate weight range of about 15% to about 60% thereof. In a particular illustrative embodiment, the product includes the water in the approximate weight of about 25% thereof.

In another embodiment, the agent to improve porosity and texture is soy protein crisp, rice crisp, or glycerine, or combinations thereof. In an illustrative embodiment, the product includes the agent to improve porosity and texture in an approximate weight range of about 4% to about 30% thereof. In a particular illustrative embodiment, the product includes soy protein crisp in the approximate weight range of about 7% to about 13% thereof. In another particular illustrative embodiment, the product includes the soy protein crisp rice in the approximate weight of about 11% thereof.

In another embodiment, the product optionally contains ammonia in an amount of about 0.5% to about 0.15%. Examples of ammonia suitable for use in the product include, but are not limited to, baker's ammonia, bicarbonate of ammonia, or ammonium bicarbonate or combinations thereof.

In one embodiment, the baking is done at an oven temperature of from about 250 .degree. F. to about 450 .degree. F., wherein the combined ingredients are placed in the oven for a time of from about 5 to about 15 minutes. In another embodiment, the baking is done at an oven temperature of about 350 .degree. F. to for a time of about 10 minutes.

In another embodiment, the invention encompasses a method of producing a toxin-decontaminant product for ingestion into the gastrointestinal tract of a patient comprising:

(a) mixing a plurality of materials including a sweetener, sugar, salt, vanilla flavoring, chocolate flavoring, activated charcoal, modified food starch, monoglycerides and sodium stearoyl lactylate;

(b) adding water to produce a mixture, for instance, a dough and mixing the mixture to produce a mixture of component materials;

(c) adding an agent to improve porosity and texture and blending the composition; and

(d) processing to remove water, for example, by baking the mixture at a predetermined temperature for a predetermined time.

In an illustrative embodiment, the activated charcoal is characterized by an internal surface area of from about 800 m²/g to about 3,000 m²/g; in another illustrative embodiment, the activated charcoal is characterized by an internal surface area of from about 1500 m²/g to about 2,500 m²/g; in yet another illustrative embodiment, the activated charcoal is characterized by an internal surface area of about 2,000 m²/g.

In another illustrative embodiment, the method includes activated charcoal in an approximate weight range of from about 10% to about 90% thereof. In another illustrative embodiment, the method includes activated charcoal in an approximate weight range of from about 20% to about 80% thereof. In another illustrative embodiment, the method includes activated charcoal in an approximate weight range of from about 25% to about 75% thereof. In another illustrative embodiment, the method includes activated charcoal in an approximate weight range of from about 30% to about 70% thereof.

In another embodiment, the flavoring agent is vanilla flavor, chocolate flavor, salt, sugar, or a sweetener. In an illustrative embodiment, the method includes vanilla flavor in the approximate weight range of about 0.01% to about 1% thereof. In yet another illustrative embodiment, the product includes the vanilla flavor in the approximate weight of about 0.03%. In another illustrative embodiment, the method includes chocolate flavor in an approximate weight range of about 0.5% to about 2% thereof. In yet another illustrative embodiment, the method includes chocolate flavor in the approximate weight of about 1% thereof. In an illustrative embodiment, the product includes salt in an approximate weight range of about 0.1% to about 1% thereof. In an illustrative embodiment, the method includes the salt in the approximate weight of about 0.1% thereof. In an illustrative embodiment, the method includes sugar in the approximate weight of about 1.5% to about 4% thereof. In yet another illustrative embodiment, the product includes the sugar in the approximate weight of about 2.5% thereof. In an illustrative embodiment, the method includes sweetener in an approximate weight range of about 0.5% to about 2% thereof. In another illustrative embodiment, the method includes a sweetener in an approximate weight range of about 0.001% to about 10% thereof. In an illustrative embodiment, the method includes a sweetener in the approximate weight of about 2% thereof; however, one of ordinary skill in the art will readily understand that the amount of sweetener used is dependent on the specific sweetener. Illustrative examples of sweeteners that can be used in an embodiment of the invention include, but are not limited to, sucrose, glucose, fructose, lactose, acesulfame-K, dextrose, sucralose, saccharin, and aspartame or neotame, or mixtures thereof.

In another embodiment, the complexing or thickening agent is sodium stearoyl lactylate or modified food starch. In an illustrative embodiment, the method includes sodium stearoyl lactylate in an approximate weight range of about 0.1% to about 2% thereof. In yet another illustrative embodiment, the method includes sodium stearoyl lactylate in the approximate weight of about 0.5% thereof. In another illustrative embodiment, the method includes modified food starch in the approximate weight range of about 0.1% to about 2% thereof. In another illustrative embodiment, the method includes the modified food starch in the approximate weight of about 0.5% thereof.

In another embodiment, the emulsifying agent is a mono- or diglyceride. In an illustrative embodiment, the method includes an emulsifying agent in the approximate weight range of about 0.1% to about 2% thereof. In yet another illustrative embodiment, the method includes the emulsifying agent in the approximate weight of about 0.5% thereof.

In another illustrative embodiment, the method includes water in the approximate weight range of about 45% to about 60% thereof. In another illustrative embodiment, the method includes the water in the approximate weight of about 53% thereof.

In another embodiment, the agent to improve porosity and texture is soy protein crisp. In an illustrative embodiment, the method includes soy protein crisp in the approximate weight range of about 4% to about 8% thereof. In another illustrative embodiment, the method includes the soy protein crisp rice in the approximate weight of about 6.6% thereof.

In one embodiment, the baking is done at an oven temperature of from about 250 .degree. F. to about 450 .degree. F., wherein the combined ingredients are placed in the oven for a time of from about 5 to about 15 minutes. In another embodiment, the baking is done at an oven temperature of about 350 .degree. F. to for a time of about 10 minutes.

In yet another embodiment, the invention encompasses a method of producing a toxin-decontaminant product for ingestion into the gastrointestinal tract of a patient comprising: mixing a plurality of materials including a sweetener, sugar, salt, vanilla flavoring, chocolate flavoring, activated charcoal, modified food starch, monoglycerides, and sodium stearoyl lactylate; adding water to produce a mixture, for instance, a dough and mixing said mixture to produce a mixture of component materials; adding soy protein rice crisps and blending the composition; and baking the mixture at a predetermined temperature for a predetermined time.

In another embodiment, the invention encompasses molding, forming or extruding the ingredients using methods known in the art to produce the product in a particular shape or size.

### Illustrative Embodiments

### Wafers Sandwiching a Cream

In one embodiment, the invention encompasses an edible product comprising a pair of biscuit-like wafers and a creamy filling sandwiched therebetween. The wafers may include coloring to make the edible product appealing and easily identified by young children in order to entice them to eat the edible product of the subject invention.

In an illustrative embodiment, a pair of disk-shaped wafers are produced. The wafers exhibit a compressed granular texture and a degree of friability akin to that of a cookie. The degree of friability is such that the wafers are easily crumbled by the average biting force generated by even a very young child. The degree of friability is also such that the crumbled wafers may thereafter be effectively disintegrated by the subsequent chewing action generated by the given young child.

Subject to the allowable ranges of their component composition weight percentages, the wafers exhibit a degree of rich, sweet flavor to accompany their cookie-like crumbly texture, wherein in an illustrative embodiment, the sweet flavor of the wafers is sufficient to encourage substantial chewing prior to ingestion into the user's gastrointestinal tract.

Each wafer includes activated charcoal, in addition to one or more flavoring agents, one or more complexing or thickening agents, one or more emulsifying agents; water, and an agent to improve porosity and texture in the approximate weight range proportions indicated in Table 2.

The combination of activated charcoal, one or more flavoring agents, one or more complexing or thickening agents, one or more emulsifying agents; water, and an agent to improve porosity and texture in the approximate weight range proportions followed by baking the ingredients results in a product having, for example, a cookie-like wafer appearance, which has a consistency emulating that of a baked cookie.

In another embodiment, to further add to the appeal and sweet flavor of the edible product, a cookie is prepared having a cream filling. In such a form, therapeutically or prophylactically effective amounts of activated charcoal are ingested by a subject who has ingested a poison or toxin in an amount sufficient to mitigate, substantially reduce, or cause the cessation of at least one adverse effect associated with the ingestion of the toxin or poison.

In another illustrative embodiment, to enhance the sweet flavor of the wafers, and to enhance the emulation of a cookie treat, a creamy white filling is sandwiched between a pair of wafers. The precise consistency, color, and taste of the filling is not integral to the invention; however, it is preferable that the filling be of a consistency and color appealing to children and that its flavor exhibit a sufficient sweet component to supplement or augment the sweet flavor of the wafers. The pleasant taste further encourages the child to likewise ingest addition “edible product of the invention,” which make up the required pharmacological activated charcoal dosage.

An important factor in the proper use of activated charcoal or any other decontaminant in toxicological treatment is, in addition to its ingestion in significant doses by the victim, the promptness with which the ingestion occurs. The edible product of the invention is such that a therapeutically or prophylactically effective amount of activated charcoal is found in the edible product and is ingested with chewing in an ordinary fashion (i.e., the way a subject would chew and swallow any other ingestible food).

In one embodiment, the activated charcoal is administered at the site at which the poisoning incident occurs, which in most cases is the victim's home, immediately following the discovery of the accidental or purposeful ingestion, before the ingested toxins have had the opportunity to be extensively absorbed into the bloodstream.

The edible product produced by the inventive method, given its inherent palatability, could easily be administered in the home or any other setting outside a medical institution, and by any individual. Hence, the edible product of the invention would not only expand the usage of activated charcoal as a decontaminant, but it would actually enhance, in a significant manner, the effectiveness of that usage.

### Leavening Agents

In the illustrative embodiments set forth above, the ingredients were blended and processed to remove water, for example by baking, in the absence of a leavening agent.

In another illustrative embodiment, the edible product contains a leavening agent to allow the edible product to rise and increase in volume. Embodiments of the invention in the form of a wafer, cracker, or pie crusts can utilize leavening to make them flaky or lighter in texture. Leavening can occur mainly during cooking, such as for pie crusts. In other cases most of the leavening happens prior to baking, as for example with many yeast breads, or more often, leavening may occur partially before and partially when the product is heated. The type of leavening used may depend on the product, for example whether the un-baked product is a batter or a dough.

Several types of leavening agents can be combined to give the maximum amount of lift to the product. For example, a recipe might require sugar and butter to be creamed, representing one type of leavening (i.e., air) and call for baking powder as well, which is another type of leavening (i.e., carbon dioxide).

In addition, the batter or dough should be suitable for holding the expanded shape, before, during, and after cooking. For example a cake containing flour will rise in the oven and hold the volume even after the cake cools and the leavening gases contract. The structure of the cake is strong enough after cooking so that the air cells remain. On the other hand, many cheesecakes and souffles which have little flour will attain a high volume in the oven but will collapse when the product is cooled. The egg protein-based walls of the little air cells are not strong enough to hold up the weight of the cake when the heated air cools and contracts.

### Cream Filling

The optional cream to be included in an embodiment of the edible product of the invention may be used to enhance the palatability and flavor of the wafers, for example, the cream may improve the texture or, mouth feel of the product. In an illustrative embodiment, a creamy white filling is sandwiched between a pair of wafers. The precise consistency, color and taste of the filling is not important to the invention. Thus, the color, texture, and its flavor should supplement or augment the sweet flavor of the wafers themselves.

In one embodiment, the optional cream filling comprises one or more flavoring agents, shortening, lecithin, salt, and sugar. In another embodiment, the optional cream filling is a reduced fat cream filling that comprises sugar, glycerine, shortening, and a fat replacer, in still another embodiment, the optional cream filling is a non-fat cream filling that comprises one or more flavoring agents, lecithin, salt, glycerine, a fat replacer, and one or more emulsifying agents.

In a particular embodiment, the flavoring agent is vanilla or chocolate flavoring. In another particular embodiment, the flavoring is vanilla flavoring, in a particular illustrative embodiment, the flavoring in the optional cream is present in an approximate weight percent of from about 0.05% to about 1%. In another particular illustrative embodiment, the flavoring in the optional cream is in an approximate weight percent of about 0.2%.

In another particular embodiment, the shortening is vegetable cubed shortening. In another particular embodiment, the shortening in the optional cream is present in an approximate weight percent of from about 25% to about 35%. In another particular illustrative embodiment, the shortening in the optional cream is in an approximate weight percent of about 31%.

In another particular embodiment, the lecithin in the optional cream is present in an approximate weight percent of from about 0.01% to about 0.5%. In another particular illustrative embodiment, the lecithin in the optional cream is present in an approximate weight percent of about 0.05%.

In another particular embodiment, the salt in the optional cream is present in an approximate weight percent of from about 0.1% to about 1%. In another particular illustrative embodiment, the salt in the optional cream is present in an approximate weight percent of about 0.25%.

In another particular embodiment, the sugar is powdered sugar. In another particular embodiment, the sugar in the optional cream is present in an approximate weight percent of from about 65% to about 75%. In another particular illustrative embodiment, the sugar in the optional cream is present in an approximate weight percent of about 69%.

In another embodiment, the fat replacer is a carbohydrate-based, protein-based, or non-digestible or non-absorbable fat-based fat replacer. Illustrative examples of fat replacers include, but are not limited to, cellulose, maltodextrins, gums, starches, fiber, polydextrose, and olestra. In another particular embodiment, the fat replacer in the optional cream is present in an approximate weight percent of from about 5% to about 15%. In another particular illustrative embodiment, the fat replacer in the optional cream is present in an approximate weight percent of about 11%.

### Therapeutic/Prophylactic Administration and Compositions

The edible products of the invention are administered to achieve efficacious levels of activated charcoal to a subject in need thereof to mitigate, cause the cessation of, or substantially reduce adverse effects associated with the ingestion of a toxin or poison.

In another embodiment encompasses treating or preventing diarrhea comprising administering to a subject in need thereof an effective amount composition of the invention comprising activated charcoal.

In another embodiment encompasses treating or preventing intestinal gas comprising administering to a subject in need thereof an effective amount composition of the invention comprising activated charcoal.

In another embodiment encompasses treating or preventing abdominal/enteric sepsis comprising administering to a subject in need thereof an effective amount composition of the invention comprising activated charcoal.

In another embodiment encompasses treating or preventing peptic ulcer disease comprising administering to a subject in need thereof an effective amount composition of the invention comprising activated charcoal.

In another embodiment encompasses treating or preventing gastritis comprising administering to a subject in need thereof an effective amount composition of the invention comprising activated charcoal.

In another embodiment encompasses treating or preventing reflux esophagitis comprising administering to a subject in need thereof an effective amount composition of the invention comprising activated charcoal.

In another embodiment encompasses treating or preventing food poisoning comprising administering to a subject in need thereof an effective amount composition of the invention comprising activated charcoal.

In another embodiment encompasses treating or preventing had breath comprising administering to a subject in need thereof an effective amount composition of the invention comprising activated charcoal.

In another embodiment encompasses treating or preventing gingivitis comprising administering to a subject in need thereof an effective amount composition of the invention comprising activated charcoal.

In another embodiment encompasses treating or preventing ostomy odor comprising administering to a subject in need thereof an effective amount composition of the invention comprising activated charcoal.

In another embodiment encompasses a method of binding dietary fat comprising administering to a subject in need thereof an effective amount composition of the invention comprising activated charcoal.

The composition of the invention is an edible product including activated charcoal and may be administered orally and chewed to allow the activated charcoal to achieve a therapeutic or prophylactic surface area. A therapeutic or prophylactic surface area is typically achieved by ordinary chewing and swallowing.

Due to the activity of the edible product of the invention, it is useful in veterinary and human medicine. As described above, the edible product of the invention is useful in mitigating, causing the cessation, or substantially reducing adverse effects associated with the ingestion of a toxin or poison.

The invention provides methods of treatment and prophylaxis by administration to a patient a therapeutically effective amount of activated charcoal comprised in an edible product of the invention. The subject may be an animal, including, but not limited, to an animal such a cow, horse, sheep, pig, chicken, turkey, quail, cat, dog, mouse, rat, rabbit, guinea pig, etc., and is preferably a mammal, and most preferably a human. In some instances, the patient is a child.

The compositions of the invention are intended to be administered orally and may be administered together with another biologically active agent. Administration can be at any time after ingestion of a toxin or poison, preferably within about 1 to about 3 hours and more preferably within about 1 hour and most preferably immediately after ingestion of a toxin or poison.

In an illustrative embodiment, it is desirable to administer the edible product of the invention locally to the gastrointestinal tract of the subject. This may be achieved, for example, and not by way of limitation, by oral administration.

The present compositions will contain a therapeutically effective amount of activated charcoal, optionally with an additional therapeutic, preferably in purified form, wherein the additional therapeutic is in a suitable amount of a pharmaceutically acceptable vehicle so as to provide the form for proper administration to the patient.

The term “vehicle” refers to a diluent, adjuvant, excipient, or carrier with which a composition of the invention is administered.

The edible product of the invention for oral delivery can also contain one or more optional agents, for example, pharmaceutical additives such as, PVP; sweetening agents such as fructose, aspartame or saccharin; flavoring agents such as peppermint, oil of wintergreen, saccharine, aspartame, neotame, or cherry; coloring agents; and preserving agents, to provide a pharmaceutically palatable preparation that do not interfere with the preparation of the edible product. In another embodiment, products of the invention may comprise additional coatings or frostings. For example, products of the invention may be enclosed in a chocolate coating as a single wafer or a filled cookie or sandwich cookie. The coating may also decrease the friable nature of the product and increase product stability or shelf life.

The amount of the decontaminant edible product of the invention that will be effective in the treatment or prevention of ingestion of a particular toxin or poison will depend on the nature of the toxin or poison, and can be readily determined by clinicians. In one embodiment, the edible product of the invention, is such that it can be administered by a parent or non-clinician, wherein such parent or non-clinician suspects that a child or animal ingested a poison or toxin.

The precise dose to be employed in the compositions will also depend on the seriousness of the toxicity or poisoning, and should be decided according to the judgment of the practitioner and each patient's circumstances. However, the edible product of the invention is such that it can be administered, to a subject suspected of ingesting a poison or toxin as one or more individual units and without worry of any adverse effect associated with administration of the edible product provided it is used appropriately.

### Oral Administration

The oral formulations of the invention may contain inert ingredients, which allow for protection against the stomach environment, and release of the biologically active material in the intestine. Such formulations, or enteric coatings, are well known in the art. For example, tablets containing a fusion protein in admixture with nontoxic pharmaceutically acceptable excipients, which are suitable for manufacture of tablets, may be used. These excipients may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc.

The therapeutically or prophylactically effective amount of edible product may be measured in a numbers of ways, including calculated to alleviate symptoms associated with a specific toxin or poison in a subject, such as the symptoms of poison or toxin ingestion.

### Package Containing Edible Product

In a further embodiment, the invention provides a package containing products of the invention. The package will typically comprise a label. Suitable packages include, for example, boxes, cellophane containers or wraps and the like. The package may be formed from a variety of materials such as cellophane or plastic. The package holds the edible product that includes activated carbon in a therapeutically or prophylactically effective amount to mitigate, cause the cessation, or substantially reduce at least one adverse effect associated with the ingestion of a poison or toxin. In addition, the edible product in the package may contain a second active agent. The label on the container typically indicates that the edible product is used for a specific therapy, and may also indicate directions for in vivo use, such as those described above.

Without further description, it is believed that a person of ordinary skill in the art can, using the preceding description and the following working examples, make and utilize the invention and practice the claimed methods. For example, a skilled artisan would readily be able to determine the administration of the edible product of the present invention. The following working examples therefore, specifically point out the illustrative embodiments of the invention, and are not to be construed as limiting in any way the remainder of the disclosure.

What is claimed is:

1. A method of treating or preventing a gastrointestinal disorder comprising administering to a subject in need thereof an edible toxin-decontaminant product comprising a plurality of ingredients said plurality comprising an effective amount of activated charcoal.

2. The method of claim 1, wherein the gastrointestinal disorder is diarrhea, intestinal gas, abdominal/enteric sepsis, peptic ulcer disease, gastritis, reflux esophagitis, food poisoning, bad breath, gingivitis, or ostomy odor.

3. The method of claim 1, wherein one or more of ingredients comprises a dough.

4. The method of claim 3, wherein the dough comprises one or more flavoring agents.

5. The method of claim 3, wherein the dough comprises one or more complexing or thickening agents or mixtures thereof.

6. The method of claim 3, wherein the dough comprises one or more emulsifying agents or mixtures thereof.

7. The method of claim 3, wherein the dough comprises an agent to improve porosity and texture.

8. The method of claim 1, wherein said activated charcoal is characterized by an internal surface area of from about 800 m²/g to about 3,000 m²/g.

9. The method of claim 1, wherein said activated charcoal is characterized by an internal surface area of about 2,000 m²/g.

10. The method of claim 1, wherein the product includes the activated charcoal in the approximate weight range of from about 20% to about 70% thereof.

11. The method of claim 1, wherein the product includes the activated charcoal in the approximate weight range of from about 25% thereof.

12. The method of claim 4, wherein the flavoring agent is vanilla flavor, chocolate flavor, salt, sugar, or a sweetener or mixtures thereof.

13. The method of claim 5, wherein the complexing or thickening agent is sodium stearoyl lactylate, high fructose corn syrup, or modified food starch or mixtures thereof.

14. The method of claim 6, wherein the emulsifying agent is a monoglyceride or diglyceride or mixtures thereof.

15. A method of binding dietary fat comprising administering to a subject in need thereof an edible toxin-decontaminant product comprising a plurality of ingredients said plurality comprising an effective amount of activated charcoal.
